(19) United States
(12) Patent Application Publication
(10) Pub. No.: US 2008/0119448 A1
(43) Pub. Date: May 22, 2008

(54) Methods of treating an ocular allergy with low dose dexamethasone

(76) Inventor: Mitchell H. Friedlaender, La Jolla, CA (US)

Correspondence Address: STERNE, KESSLER, GOLDSTEIN & FOX P.L.L.C., 1100 NEW YORK AVENUE, N.W., WASHINGTON, DC, 20005, US

(21) Appl. No.: 11/979,356

(22) Filed: Nov. 1, 2007

Related U.S. Application Data
(60) Provisional application No. 60/856,026, filed on Nov. 2, 2006.

(51) Int. Cl. A61K 31/573

(52) U.S. Cl. 514/179

(57) ABSTRACT

The present invention is directed to a method of treating an ocular allergy with a composition comprising a low dose of dexamethasone. In some embodiments, the present invention is directed to a method of treating an ocular allergy, the method comprising administering to an ocular surface of a subject in need thereof a composition comprising about 0.001% to about 0.01% (w/v) dexamethasone.

This application claims the benefit of the filing date of U.S. appl. Ser. No. 60/856,026, filed Nov. 2, 2006, the entirety of which is fully incorporated by reference herein.

### BACKGROUND OF THE INVENTION

1. Field of the Invention

The present invention is directed to a method of treating an ocular allergy with a composition comprising a low dose of dexamethasone. In some embodiments, the present invention is directed to a method of treating an ocular allergy, the method comprising administering to an ocular surface of a subject in need thereof a composition comprising about 0.001% to about 0.01% (w/v) dexamethasone.

2. Background Art

Allergic conjunctivitis is a common ocular condition, affecting by some estimates about 15% of the general population. Since ocular allergic symptoms often accompany nasal allergic symptoms, the condition is sometimes referred to as “allergic rhinoconjunctivitis.” Allergies include seasonal allergies, perennial allergies and hereditary allergies. Seasonal allergies can be triggered by airborne pollens, such as those from trees, grasses, and weeds. Perennial allergic rhinoconjunctivitis occurs throughout the year, and may be triggered by animal dander, dust, molds, and feathers.

Ocular signs and symptoms of allergies include itching, redness, swelling, tearing, burning and stinging. Treatment of ocular allergy with eye drops includes over the counter antihistamine/vasoconstrictor eye drops (e.g., Opcon-A® (Bausch & Lomb, Rochester, N.Y.), Naphcon-A® (Alcon, Fort Worth, Tex.), and Vasocon-A® (Novartis Ophthalmics, East Hanover, N.J.)), and prescription mast cell stabilizers (e.g., Patanol® (Alcon, Fort Worth, Tex.), Zaditor® (Novartis Ophthalmics, East Hanover, N.J.), Elestat® (Inspire Pharmaceuticals, Durham, N.C.), Optivar® (Medpointe Pharmaceuticals, Somerset, N.J.), Alamast® (Vistakon Pharmaceuticals, Jacksonville, Fla.), and Alocril® (Allergan, Irvine, Calif.). One nonsteroidal anti-inflammatory agent, ketorolac tromethamine (Acular LS®, Allergan, Irvine, Calif.) has FDA approval for the treatment of allergic conjunctivitis.

Corticosteroid eye drops are more effective in treating an ocular allergy than either vasoconstrictor/antihistamines, mast cell stabilizers, or nonsteroidal anti-inflammatory eye drops. However, none of the above listed commercial eye drops comprise corticosteroids. In some instances, corticosteroids have been shown to have undesirable ocular side effects, such as elevation of intraocular pressure, cataract formation, and opportunistic infections. A commercial eye drop comprising higher amounts of corticosteroids is available available under the tradename Alrex® (lotoprednol etabonate 0.2%; Bausch & Lomb, Rochester, N.Y.), which is FDA approved for the treatment of ocular allergy.

There exists a need in the art for a corticosteroid product sufficient for treatment of an ocular allergy, wherein the corticosteroid product has reduced undesirable side effects.

### BRIEF SUMMARY OF THE INVENTION

The present invention is directed to a method of treating an ocular allergy, the method comprising administering to an ocular surface of a subject in need thereof a composition comprising about 0.001% to about 0.01% (w/v) dexamethasone.

In some embodiments, the composition comprises about 0.005% to about 0.01% (w/v) dexamethasone. In some embodiments, the composition comprises about 0.005% (w/v) dexamethasone.

In some embodiments, the composition is administered in a liquid state.

In some embodiments, the administering does not result in an increase in intraocular pressure.

In some embodiments, the composition further comprises a tonicity agent, a pH adjuster, a preservative, a demulcent, a buffering agent, a lubricant, or combinations thereof.

In some embodiments, the tonicity agent is selected from the group consisting of dextrose, sodium chloride, calcium chloride, potassium chloride, magnesium chloride, boric acid, and combinations thereof.

In some embodiments, the pH adjuster is selected from the group consisting of hydrochloric acid, acetic acid, sodium hydroxide, potassium hydroxide, an alkali earth metal hydroxide, an alkaline earth metal hydroxide, an organic base, an organic acid, and combinations thereof.

In some embodiments, the preservative is selected from the group consisting of benzalkonium chloride, ethylenediaminetetraacetic acid and salts thereof, Purite®, chlorobutanol, sodium perborate, sorbic acid, and combinations thereof.

In some embodiments, the demulcent is selected from the group consisting of a soluble cellulose derivative, dextran, gelatin, polyol, polyvinyl alcohol, povidone, chondroitin sulfate, hyaluronic acid, and combinations thereof.

In some embodiments, the buffering agent is selected from the group consisting of citric acid, sodium citrate, boric acid, sodium borate, one or more sodium salts of phosphoric acid, one or more potassium salts of phosphoric acid, sodium bicarbonate, and combinations thereof.

In some embodiments, the lubricant is selected from the group consisting of dextran, hydroxypropyl methylcellulose (HPMC), glycerin, polyethylene glycol (PEG) and propylene glycol.

In some embodiments, the dexamethasone is dexamethasone sodium phosphate.

In some embodiments, the composition comprises: (a) dexamethasone in a concentration of about 0.001% to about 0.01% (w/v); (b) a preservative; (c) a tonicity agent; and (d) a pH adjuster.

In some embodiments, the preservative is benzalkonium chloride.

In some embodiments, the tonicity agent is sodium chloride.

In some embodiments, the pH adjuster is sodium hydroxide.

In some embodiments, the composition comprises: (a) dexamethasone in a concentration of about 0.001% to about 0.01% (w/v); (b) the preservative is benzalkonium chloride at a concentration of about 0.01% to 0.02% (w/v); (c) the tonicity agent is sodium chloride at a concentration of about 0.5% to about 1.5% (w/v); and (d) the pH adjuster is sodium hydroxide in an amount sufficient to bring the pH of the composition to about 4.0 to about 7.0.

In some embodiments, the present invention is directed to a kit comprising: (a) a composition comprising about 0.001% to about 0.01% (w/v) dexamethasone; and (b) instructions for using the composition of (a).

In some embodiments, the kit further comprises a means for administering the composition.

In some embodiments, the present invention is directed to a kit comprising: (a) a composition comprising about 0.001% to about 0.01% (w/v) dexamethasone, wherein the composition is individually packaged for a single administration; and (b) instructions for using the composition of (a). In some embodiments, the composition of the kit does not comprise a preservative.

### DETAILED DESCRIPTION OF THE INVENTION

The present invention is directed to a method of treating an ocular allergy with a composition comprising a low dose of dexamethasone. In some embodiments, the present invention is directed to a method of treating an ocular allergy, the method comprising administering to an ocular surface of a subject in need thereof a composition comprising about 0.001% to about 0.01% (w/v) dexamethasone.

It is to be noted that the term “a” or “an” refers to one or more of that entity; for example, “a tonicity agent,” is understood to represent one or more tonicity agents. As such, the terms “a” (or “an”), “one or more,” and “at least one” can be used interchangeably herein. As used herein, “about” refers to plus or minus 10% of the indicated number. For example, “about 0.5%” indicates a range of 0.45% to 0.55%.

The compositions of the present inventions are “ophthalmically acceptable.” The term “ophthalmically acceptable” refers to those compounds, materials, compositions, and/or solutions which are, within the scope of sound medical judgment, suitable specifically for contact with the tissues of the eye, and the area surrounding the eye without excessive toxicity, irritation, allergic response, or other complications commensurate with a reasonable benefit/risk ratio.

The term “dexamethasone,” also known as dexamethazone, refers to a corticosteroid of Formula I, or derivatives, salts or esters thereof.

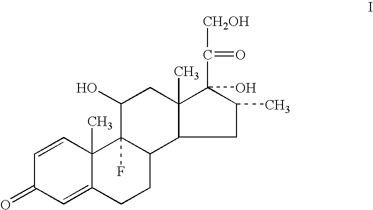

Thus, the term dexamethasone includes, but is not limited to, e.g., dexamethasone sodium phosphate, dexamethasone (alcohol), dexamethasone acetate, dexamethasone dimethylbutyrate, dexamethasone trimethylacetate, dexamethasone dipropionate, dexamethasone acefurate, and mixtures thereof.

In the present invention, the composition comprises low concentrations of dexamethasone. Traditionally, these low concentrations of dexamethasone would not be expected to produce any corticosteroid-related side-effects. Shulman, D. G. et al., Opthamol. 106:362-9 (1999). However, numerous studies have shown that administration of dexamethasone at 0.1% results in an increase in intraocular pressure. See e.g., Tripathi, R. C. et al., Drugs and Aging, 15:439-50 (1999). The present invention provides a method of administering dexamethasone at a concentration that does not result in an increase in intraocular pressure, yet still provides effective treatment for an ocular allergy. In some embodiments, the composition comprises about 0.0005% to about 0.05% (w/v), 0.001% to about 0.02% (w/v), or about 0.005% to about 0.01% (w/v) dexamethasone. In some embodiments, the composition comprises about 0.0025% to about 0.005% (w/v) dexamethasone.

In some embodiments, the composition can further comprise an antihistamine (e.g., antazoline phosphate, pheniramine maleate), vasoconstrictor (e.g., naphazoline hydrochloride), mast cell stabilizer (e.g., olopatadine, ketotifen, epinastine, azelastine, pemirolast, and nedocromil), nonsteroidal anti-inflammatory agent (e.g., ketorolac tromethamine), or combinations thereof.

The present compositions can be in any physical state suitable to be administered to the eye. Such physical forms include, but are not limited to, liquids (e.g., solutions or suspensions), semi-solids (gels, creams, ointments, etc.), and the like. Each of these physical states of the present compositions can be prepared using techniques and processes which are conventional and well known in the art. For a more detailed discussion of the preparation and administration of ophthalmic formulations see Remington's Pharmaceutical Sciences, 15 Ed., 1489-1504 (1975) which is incorporated in its entirety herein by reference. In some embodiments, the composition is administered in the state of a liquid.

The term intraocular pressure refers to the fluid pressure inside the eye. Intraocular pressure can be measured in various ways known to those in the art, e.g., intraocular pressure can be measured with a tonometer. Normal eye pressure, as measured by an eye doctor, usually ranges between 10 and 21 mm of mercury, with an average of 16. Thus, in some embodiments the present invention is directed to a method of treating an ocular allergy with a composition comprising dexamethasone, wherein after administration the intraocular pressure remains between 10 and 21 mm of mercury as measured by a tonometer. Intraocular pressure that is consistently above 21 indicates ocular hypertension. Ocular hypertension can develop into glaucoma, a serious disease that causes damage to the optic nerve. Thus, in some embodiments of the present invention is provided a method of treating an ocular allergy with a composition comprising dexamethasone, wherein there is not an increased risk of ocular hypertension or glaucoma.

The term “ocular surface” refers to eye ball surface as well as the external skin surrounding the eye, i.e., the eyelid and the margin of the eyelid, and associated hair projecting therefrom, i.e., eyelashes and eye brows. The ocular surface can be present on any animal having an eyelid. Thus, methods of the present invention are applicable to both human use and veterinary use, preferably for human use. In some embodiments, the methods are applicable for use on domesticated animals such as companion animals (dogs and/or cats), livestock, or other economically important animals (e.g., model or breeding animals).

The term “ocular allergy” refers to a sensitivity to allergens that, under normal conditions, are considered innocuous to others. The terms “ocular allergy” includes allergic conjunctivitis and allergic rhinoconjuctivitis. An ocular allergy includes inherited allergies as well as seasonal and perennial allergies. Symptoms include itching, redness, swelling, burning sensation, watery eyes (excess tearing), or sometimes mild mucoid discharge from the eye. An ocular allergy can affect one or both eyes of the subject being affected. Symptoms associated with ocular allergies include itching, swelling, redness, tearing and burning of the ocular area.

The term “treating” or “treatment” refer to both therapeutic treatment and prophylactic or preventative measures, wherein the object is to prevent, inhibit, reverse or slow down (lessen) any undesired effect of the ocular allergy. For purposes of this invention, beneficial or desired results include, but are not limited to, alleviation of symptoms; diminishment of extent of the ocular allergy, stabilized (i.e., not worsening) state of the ocular allergy, delay in onset of the ocular allergy, or slowing of progression of the ocular allergy; amelioration of the ocular allergy, remission of the ocular allergy (whether partial or total); or enhancement or improvement of the symptoms of the ocular allergy.

The method of the present invention can comprise administering the composition once daily or more than once daily (e.g., twice daily or three times per day). In some embodiments, the present invention can be administered on an “as needed” basis or an “as desired” basis. In some embodiments, the composition can be administered once daily to achieve relief from the symptoms of the ocular allergy. Single daily administration can be beneficial for various reasons, e.g., single daily administration can result in greater convenience and higher patient compliance.

Various amounts of the composition can be administered to an ocular surface.

One of skill in the art will understand that the amount to be administered is dependent on various factors, e.g., the location of administration, the concentration of the dexamethasone, the viscosity of the composition, the frequency of administration, severity of the allergy, etc. In some embodiments, the amount of the composition to be administered is less than about 10 mL, about 0.01 mL to about 5 mL, about 0.1 mL to about 3 mL, or about 0.2 mL to about 1 mL. In embodiments wherein the composition is a liquid, the amount of the composition to be administered can vary, e.g., 1 to 20 drops, 1 to 10 drops, or 2 to 5 drops.

In some embodiments, the composition further comprises a tonicity agent, a pH adjuster, a preservative, a demulcent, a buffering agent, a lubricant, or combinations thereof.

Various tonicity agents can be used. Tonicity agents can be used to adjust the salt concentration of the composition, provided the agent is ophthalmically acceptable. In some embodiments, the tonicity agent is selected from the group consisting of dextrose, sodium chloride, calcium chloride, potassium chloride, magnesium chloride, boric acid, and combinations thereof. In some embodiments, the tonicity agent is used to produce an isotonic composition. In some embodiments, the tonicity agent is used to produce a hypotonic composition.

Various pH adjusting agents can be used in the present invention. pH adjusting agents include any composition or compound capable of adjusting the pH of the composition to the desired level. pH adjusting agents can include acids and/or bases. In some embodiments, the pH adjusting agent is selected from the group consisting of hydrochloric acid, acetic acid, sodium hydroxide, potassium hydroxide, an alkali earth metal hydroxide, an alkaline earth metal hydroxide, an organic base, an organic acid, and combinations thereof. One of skill in the art can determine the appropriate concentrations and amounts of the pH adjusting agent suitable for use when applying to an ocular area.

In some embodiments, the pH adjusting agent can be included to provide the compositions of the present invention at a pH in a physiologically acceptable range, e.g., in a pH range of about 3 to about 9, about 4 to about 8.5, about 5 to about 8.5, or about 5.0 to about 7.0. In some embodiments, the pH adjusting agent provides and/or maintains a pH of about 4.0 to about 7.0 or about 5.0 to about 6.0. As one of skill in the art will recognize, the pH can vary over time, depending on various factors, e.g., stability of the composition, amount of exposure to the atmosphere, etc. Thus, as used herein, when referring to compositions, kits or methods of the present invention, any specified pH refers to the pH at any time between the time of manufacturing and time of administering. In some embodiments, the composition remains in a slightly acidic pH, since dexamethasone is more stable in a slightly acidic pH.

Acids useful as pH adjusting agents in the present compositions can include boric acid, hydrochloric acid, acetic acid, organic acids, other acids which are ophthalmically acceptable in the concentrations and pH ranges used, and the like.

Bases useful as pH adjusting agents in the present compositions include, but are not limited to, sodium and/or potassium hydroxides, other alkali and/or alkaline earth metal hydroxides, organic bases, other bases which are ophthalmically acceptable in the concentrations and pH levels used, and the like.

In some embodiments of the present invention the composition comprises a preservative. The term “preservative” refers to any substance that can keep the dexamethasone chemically stable, can slow or retard the degradation or alteration of dexamethasone, and/or can inhibit the growth of microorganisms in the composition. For example, a preservative can protect the dexamethasone from instability caused by light, moisture, heat, or oxidation. In some embodiments, the preservative can be an antioxidant. In some embodiments, the preservative can be an antimicrobial. Preservatives include, but are not limited to, α-lipoic acid, α-tocopherol, ascorbyl palmitate, benzyl alcohol, biotin, bisulfites, butylated hydroxyanisole (BHA), butylated hydroxytoluene (BHT), ascorbic acid and its esters, carotenoids, calcium citrate, acetyl-L-carnitine, chelating agents, chondroitin, citric acid, coenzyme Q-10, cysteine, cysteine hydrochloride, 3-dehydroshikimic acid (DHS), EDTA (ethylenediaminetetraacetic acid) and salts thereof, ferrous sulfate, folic acid, fumaric acid, niacin, tocopherol and their esters, e.g., tocopherol acetate, tocopherol succinate, tocotrienal, d-α-tocopherol acetate, vitamin A and its esters, vitamin B and its esters, vitamin C and its esters, vitamin D and its esters, vitamin E and its esters, e.g., vitamin E acetate, and combinations thereof. In some embodiments, the preservative is selected from the group consisting of benzalkonium chloride, EDTA, Purite® (Allergan, Irvine, Calif.), chlorobutanol, sodium perborate, sorbic acid, and combinations thereof.

Various concentrations of preservative can be used in the composition of the invention, dependent on, e.g., the type of preservative, its mechanism of action, and/or its ophthalmic acceptability. In some embodiments, the preservative is about 0.00001% to about 4.0% w/v of the composition, or about 0.0001% to about 1.0% w/v of the composition, or about 0.001% to about 0.1% w/v of the composition.

Various demulcents can be used. The term “demulcent” refers to any compound or composition that when applied to an ocular surface can lubricate, soothe and/or protect the mucous membrane of the eye.

In some embodiments, the demulcent is selected from a water soluble cellulose derivative, polyethylene glycol, polyvinyl alcohol, dextran, gelatin, polyol, polyvinyl alcohol, povidone, chondroitin sulfate, carbomer, tragacanth, hyaluronic acid, colloidal magnesium aluminum silicate, sodium alginate, and combinations thereof. Various water soluble cellulose derivatives can be used. Examples include, but are not limited to, carboxymethylcellulose, one or more salts of carboxymethylcellulose, hydroxyethyl cellulose, hypromellose, methylcellulose, and combinations thereof. The term “polyol” refers to a compound with greater than 2 alcohol groups. Examples of polyols include, but are not limited to glycerin, polyethylene glycol, polysorbate, propylene glycol, and combinations thereof. Various concentrations of demulcents can be used in the present invention. In some embodiments, the demulcent is about 0.01% to about 20.0% w/v of the composition, about 0.1% to about 10.0% w/v of the composition, or about 0.5% to about 5.0% w/v of the composition.

In some embodiments, the composition is water soluble. The term “water soluble” refers the ability of the composition to dissolve (i.e., form a solution) in water. One of skill in the art will recognize that solubility will be dependent on the volume of the solvent (i.e., water), the presence or absence of other compounds (e.g., solubilizing agents), the temperature of the solvent, as well as other factors. In some embodiments, the term “water soluble” refers to the ability of at least 90% of a substance to dissolve completely in water at room temperature in 1 hour, wherein the substance is 10% the volume of the water.

In some embodiments, the composition comprises a buffering agent. The terms “buffer” or “buffering agent” refer to an ophthalmically acceptable compound or composition that can neutralize both acids and bases, thereby maintaining the original acidity or basicity of the composition. Buffers can include, but are not limited to, phosphate buffers (e.g., sodium and potassium phosphates), phosphates, bicarbonate, citrate, borate, acetate buffers, citrate buffers, tromethamine buffers and combinations thereof. Preferred buffers are selected from the group consisting of citric acid, sodium citrate, boric acid, sodium borate, one or more sodium salts of phosphoric acid, one or more potassium salts of phosphoric acid, sodium bicarbonate, and combinations thereof.

In some embodiments, the composition of the invention further comprises a lubricant. In some embodiments, the lubricant is selected from the group consisting of dextran, hydroxypropyl methylcellulose (HPMC), glycerin, polyethylene glycol (PEG) and propylene glycol.

In some embodiments, the composition of the present invention is substantially free of lipids. Compositions comprising lipids can cause blurred vision when applied to a subject suffering from ocular allergies. The term “lipid” refers to hydrocarbon-based molecules of biological origin that are predominantly nonpolar or hydrophobic. The basic classes of lipids are: fatty acids (e.g., saturated or unsaturated fatty acids), glycerides or glycerolipids (e.g., monoglycerides, diglycerides, triglycerides (neutral fats), phosphoglycerides or glycerophospholipids), nonglycerides (e.g. sphingolipids, sterol lipids (includes cholesterol and steroid hormones)), prenol lipids (includes terpenoids), waxes, polyketides, and complex lipid derivatives (e.g., glycolipids, and protein-linked lipids). In some embodiments, the term lipid refers to petrolatum or petroleum. The term “petrolatum” refers to a substance which is a complex combination of semi-solid, saturated hydrocarbons, mainly of a paraffinic nature, obtained from petroleum. Generally, petrolatum comprises liquid hydrocarbons having carbon numbers predominately greater than C₂₅.

In some embodiments, the term “substantially free of lipids” refers to a composition wherein about 0% to 2% (w/w) of the composition is a lipid, about 0% to 1% (w/w) of the composition is a lipid, about 0% to 0.5% (w/w) of the composition is a lipid, or about 0% to 0.2% (w/w) of the composition is a lipid, or about 0% to 0.1% (w/w) of the composition is a lipid. In some embodiments, the term “substantially free of lipids” refers to compositions wherein less than 0.1% of the composition is a lipid.

In some embodiments, the composition comprises: (a) dexamethasone in a concentration of about 0.001% to about 0.01% (w/v); (b) a preservative; (c) a tonicity agent; and (d) a pH adjuster. In some embodiments, the preservative is benzalkonium chloride, the tonicity agent is sodium chloride, and the pH adjuster is sodium hydroxide.

In some embodiments, the composition comprises: (a) dexamethasone in a concentration of about 0.001% to about 0.01% (w/v); (b) the preservative is benzalkonium chloride at a concentration of about 0.01% to 0.02% (w/v); (c) the tonicity agent is sodium chloride at a concentration of about 0.5% to about 1.5% (w/v); and (d) the pH adjuster is sodium hydroxide in an amount sufficient to bring the pH of the composition to about 4.0 to about 7.0.

In some embodiments, the present invention is directed to a kit comprising: (a) a composition comprising about 0.001% to about 0.01% (w/v) dexamethasone; and (b) instructions for using the composition of (a). In a related embodiment, the present invention can be directed to a kit comprising: (a) a composition comprising about 0.001% to about 0.01% (w/v) dexamethasone, wherein the composition is individually packaged for a single administration; and (b) instructions for using the composition of (a).

In some embodiments, the kit further comprises a means for administering the composition, i.e., an applicator. In some embodiments, the means for administering can include a bottle, dropper, eye-wash apparatus, wetted towel, wetted swab, or sponge.

The composition can be individually packaged, e.g., in a bottle, jar, ampoule, tube, syringe, envelope, container, or vial. When the composition is individually packaged, in some embodiments the composition does not comprise a preservative. Alternatively, the composition can be contained in a package that is capable of holding multiple units, e.g., in resealable glass or plastic packages. In some kits, the components of the composition are mixed together immediately preceding their usage. For example, in some embodiments one or more dry components of the composition of the kit are packaged in a separate container, e.g., a plastic bottle, and then mixed with one or more of the liquid components of the composition immediately prior to use. Optionally, the kit of the present invention can include a dropper or other device for transferring/administering the composition to a subject.

Optionally, the kit can further comprise printed matter containing instructions for using the composition of the present invention. For example, such printed instructions can be in a form prescribed by a governmental agency regulating the manufacture, use or sale of pharmaceuticals or biological products, which reflects approval by the agency of the manufacture, use or sale for human application. In some embodiments, the kit further comprises printed matter, which, e.g., provides information on the use of the composition or a pre-recorded media device which, e.g., provides information on the use of the method of the present invention.

“Printed matter” can be, for example, a book, booklet, brochure, leaflet or the like. The printed matter can describe the use of the composition of the present invention. Possible formats include, but are not limited to, a bullet point list, a list of frequently asked questions (FAQ) or a chart. Additionally, the information to be imparted can be illustrated in non-textual terms using pictures, graphics or other symbols.

The kit can also include a container for storing the components of the kit. The container can be, for example, a bag, box, envelope or any other container that would be suitable for use in the present invention. In some embodiments, the container is large enough to accommodate each component of the present invention. However, in some cases, it can be desirable to have a smaller container which is large enough to carry only some of the components of the present invention.

### EXAMPLES

A 0.005% dexamethasone composition was prepared as outlined in Table 1.

| Ingredient | Percent, w/v |
|---|---|
| Dexamethasone Sodium Phosphate | 0.005 |
| Benzalkonium Chloride | 0.015 |
| Sodium Chloride | 0.9% |
| Sodium Hydroxide | pH 6-7 |
| Purified water | qs 100 |

### Example 2

The efficacy of the dexamethasone composition of Example 1 was compared to the commercial product Patanol® (Alcon Laboratories, Fort Worth, Tex.). Eleven subjects presenting with symptoms of an ocular allergy were treated with the dexamethasone composition of Example 1 in one eye, and with Patanol® in the other eye, twice daily for two weeks. Two weeks after treatment was initiated, subjects were examined by a medical care provider or interviewed by phone. Of the eleven subjects, five found the dexamethasone drops superior to Patanol® for relief of redness, itching and swelling; four subjects found Patanol® superior, and one found the two compositions equally effective. One subject did not provide feedback.

It is to be appreciated that the Detailed Description section, and not the Summary and Abstract sections, is intended to be used to interpret the claims. These examples illustrate possible compositions used in the present invention. While the invention has been particularly shown and described with reference to some embodiments thereof, it will be understood by those skilled in the art that they have been presented by way of example only, and not limitation, and various changes in form and details can be made therein without departing from the spirit and scope of the invention. Thus, the breadth and scope of the present invention should not be limited by any of the above-described exemplary embodiments, but should be defined only in accordance with the following claims and their equivalents.

All documents cited herein, including journal articles or abstracts, published or corresponding U.S. or foreign patent applications, issued or foreign patents, or any other documents, are each entirely incorporated by reference herein, including all data, tables, figures, and text presented in the cited documents.

What is claimed is:

1. A method of treating an ocular allergy, the method comprising administering to an ocular surface of a subject in need thereof a composition comprising about 0.001% to about 0.01% (w/v) dexamethasone.

2. The method of claim 1, wherein said composition comprises about 0.005% to about 0.01% (w/v) dexamethasone.

3. The method of claim 1, wherein said composition comprises about 0.005% (w/v) dexamethasone.

4. The method of claim 1, wherein said composition is administered in the state of a liquid.

5. The method of claim 1, wherein said administering does not result in an increase in intraocular pressure.

6. The method of claim 1, wherein said composition further comprises a tonicity agent, a pH adjuster, a preservative, a demulcent, a buffering agent, a lubricant, or combinations thereof.

7. The method of claim 6, wherein said tonicity agent is selected from the group consisting of dextrose, sodium chloride, calcium chloride, potassium chloride, magnesium chloride, boric acid, and combinations thereof.

8. The method of claim 6, wherein said pH adjuster is selected from the group consisting of hydrochloric acid, acetic acid, sodium hydroxide, potassium hydroxide, an alkali earth metal hydroxide, an alkaline earth metal hydroxide, an organic base, an organic acid, and combinations thereof.

9. The method of claim 6, wherein said preservative is selected from the group consisting of benzalkonium chloride, ethylenediaminetetraacetic acid or salts thereof, Purite®, chlorobutanol, sodium perborate, sorbic acid, and combinations thereof.

10. The method of claim 6, wherein said demulcent is selected from the group consisting of a soluble cellulose derivative, dextran, gelatin, polyol, polyvinyl alcohol, povidone, chondroitin sulfate, hyaluronic acid, and combinations thereof.

11. The method of claim 6, wherein said buffering agent is selected from the group consisting of citric acid, sodium citrate, boric acid, sodium borate, one or more sodium salts of phosphoric acid, one or more potassium salts of phosphoric acid, sodium bicarbonate, and combinations thereof.

12. The method of claim 6, wherein said lubricant is selected from the group consisting of dextran, hydroxypropyl methylcellulose, glycerin, polyethylene glycol, and propylene glycol.

13. The method of claim 1, wherein the dexamethasone is dexamethasone sodium phosphate.

14. The method of claim 1, wherein the composition comprises:
(a) dexamethasone in a concentration of about 0.001% to about 0.01% (w/v);
(b) a preservative;
(c) a tonicity agent; and
(d) a pH adjuster.

15. The method of claim 14, wherein the preservative is benzalkonium chloride.

16. The method of claim 14, wherein the tonicity agent is sodium chloride.

17. The method of claim 14, wherein the pH adjuster is sodium hydroxide.

18. The method of claim 14, wherein the composition comprises:
(a) dexamethasone in a concentration of about 0.001% to about 0.01% (w/v);
(b) the preservative is benzalkonium chloride at a concentration of about 0.01% to 0.02% (w/v);
(c) the tonicity agent is sodium chloride at a concentration of about 0.5% to about 1.5% (w/v); and
(d) the pH adjuster is sodium hydroxide in an amount sufficient to bring the pH of the composition to about 4.0 to about 7.0.

19. A kit comprising:
(a) a composition comprising about 0.001% to about 0.01% (w/v) dexamethasone; and
(b) instructions for using the composition of (a).

20. The kit of claim 18, further comprising a means for administering the composition.

21. A kit comprising:
(a) a composition comprising about 0.001% to about 0.01% (w/v) dexamethasone, wherein the composition is individually packaged for a single administration; and
(b) instructions for using the composition of (a).

22. The kit of claim 21, wherein the composition does not comprise a preservative.
